(19) United States
(12) Patent Application Publication
(10) Pub. No.: US 2020/0109163 A1
(43) Pub. Date: Apr. 9, 2020

(54) CARBONATED BEVERAGE AND METHOD FOR CARBONATION ENHANCEMENT

(71) Applicant: INTERNATIONAL FLAVORS & FRAGRANCES INC., New York, NY (US)

(72) Inventors: Xiao-Qing TANG, Holmdel, NJ (US); Jung-A KIM, Edgewater, NJ (US); Richard GOMEZ, South River, NJ (US); Asha JAJA-CHIMEDZ, North Brunswick, NJ (US); Roberto SALAS SANTOS, Lawrenceville, NJ (US); Hou WU, East Brunswick, NJ (US); Xiaodong LI, Middletown, NJ (US); Thumpalasseril V. JOHN, Morganville, NJ (US)

(21) Appl. No.: 16/152,960

(22) Filed: Oct. 5, 2018

(51) Int. Cl. C07J 9/00; A23L 2/54; A23L 2/56; A23L 27/20; A23L 27/00; C12G 3/06; C07J 71/00; C07J 41/00; C07J 17/00

(52) U.S. Cl. CPC C07J 9/00; A23L 2/54; A23L 2/56; A23L 27/203; A23V 2002/00; C12G 3/06; C07J 71/0005; C07J 41/0061; C07J 17/005; A23L 27/88

(57) ABSTRACT

Compositions and methods for enhancing carbonation effect in food consumables are provided.

### INTRODUCTION

This application is a Continuation-in-Part application of U.S. application Ser. No. 16/083,641, filed Sep. 10, 2018, which is a National Phase Application of PCT/US2017/019903, filed Feb. 28, 2017, which claims benefit of priority from U.S. Provisional Application Ser. No. 62/302,418, filed Mar. 2, 2016 and 62/324,385, filed Apr. 19, 2016, the contents of which are incorporated herein by reference in their entireties.

### BACKGROUND

There is an ongoing need in the flavor industry for taste modifying compounds that improve, enhance or modify flavors for food preparations. Those with skill in the art appreciate how differences in the chemical structures of the molecules can result in significant differences in functions. The identification of structural variations and discovery of new compounds enable the creation of new flavors.

Phytosterols occur in plants and encompass sterols and stanols. Stanols are saturated forms of corresponding sterols. Phytosterols are steroid compounds similar to cholesterol. They are poorly absorbed and can compete with cholesterol for absorption in the intestine, resulting lower levels of cholesterol. Phytosterol-enriched foods and dietary supplements have been marketed for decades. Phytosterols are also disclosed as functional ingredients to supplement food and beverages especially sweetener compositions to promote health and wellness (U.S. Pat. Nos. 6,129,944 and 9,131,720). A naturally occurring phytosterol blend CHOLESTATIN® is reported to provide enhanced flavorings such as vanilla, chocolate, butter, cheese, strawberry, raspberry, blueberry, orange, lemon, apple, grape, lemon-lime, lime, watermelon, coconut, beef, bacon, chicken, pork, onion, garlic, pepper, ranch, nacho, taco, cheddar, romano, parmesan, cream, buttermilk, blue cheese and combinations thereof, and is therefore proposed to be incorporated in a wide variety of foods including popcorn, baked goods, cheese sauce, dips, condiments, dressings, marinades, fillings, toppings, snack blends and side dishes, cereals, yogurt, fried foods, prepared meals, dairy products, frostings, gravies, ice cream, snacks and chips, crackers, puddings, candies and nutritional bars (US Publication No. 2005/0064078).

More than 200 sterols and related compounds have been identified (Akhisa, et al. (1991) In: Physiology and Biochemistry of Sterols, Patterson, G. W. and W. D. Nes (Eds.). American Oil Chemists Society, Champaign, Ill., 172-228). However, available sterols exist mostly as mixtures and the reported separations are laborious (Zhang, et al. (2005) Steroids, 70(13): 886-895). In the flavor industry, none of the individual sterols has been isolated, investigated and conclusively characterized.

### SUMMARY OF THE INVENTION

This invention provides a method of enhancing carbonation effect in a carbonated beverage using an olfactory effective amount of a compound of Formula I:

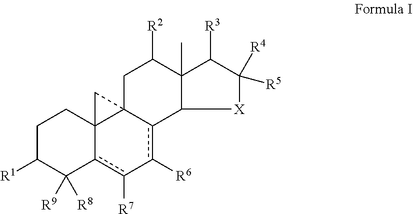

wherein
X is —C— or —C—C—;
R¹ is —OH or —O—R¹⁰;
R² and R⁶ are independently —H or —OH;
   R³ is a substituted or unsubstituted C₁-C₁₂ alkyl, substituted or unsubstituted C₂-C₁₂ alkenyl, or substituted or unsubstituted heterocycloalkyl group,
   R⁴ is —H or —OH, or
   R³ and R⁴ taken together form a substituted or unsubstituted 5- or 6-membered cycloalkyl, heterocycloalkyl or spiro-heterocycloalkyl ring;
R⁵ is —H, —CH₂OH or —COOH;
R⁷ is —H, —OH or —O—R¹⁰;
R⁸ and R⁹ are independently —H or —CH₃;
R¹⁰ is a —C(═O)CH₃, or substituted or unsubstituted cycloalkyl or heterocycloalkyl group; and
dashed bonds are independently present or absent.

In some aspects, this invention provides a method of enhancing carbonation effect in a carbonated beverage using an olfactory effective amount of a compound of Formula II:

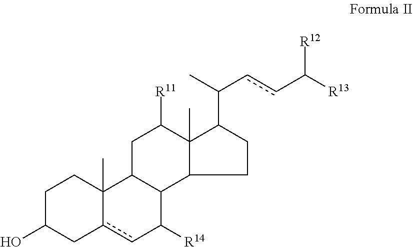

wherein
R¹¹ and R¹⁴ are independently —H or —OH;
R¹² is a lower alkyl or oxo group;
R¹³ is a lower alkyl, hydroxyl, or amine group; and
dashed bonds are independently present or absent. In certain embodiments, the compound is β-sitosterol, stigmasterol, chenodeoxycholic acid or CHAPS. In other embodiments, the olfactory effective amount is about 1 part per trillion or greater by weight, or more preferably about 1 part per trillion to about 100 parts per billion by weight. In certain embodiments, the carbonated beverage is selected from the group consisting of a carbonated water, a mineral water, a soda, a beer, a sparkling wine and a Champagne. A carbonated beverage including an olfactory effective amount of a compound of Formula I, or more preferably a compound of Formula II, or most preferably β-sitosterol, stigmasterol, chenodeoxycholic acid or CHAPS is also provided.

### DETAILED DESCRIPTION OF THE INVENTION

It has now unexpectedly been found that sitosterol enhances and its analogs enhance the carbonation effect in carbonated beverages. Accordingly, the present invention provides methods for enhancing carbonation effect in carbonated beverages using a sitosterol or structural analog thereof.

A sitosterol or structural analog of sitosterol of use in the methods of this invention has the general structure of Formula I.

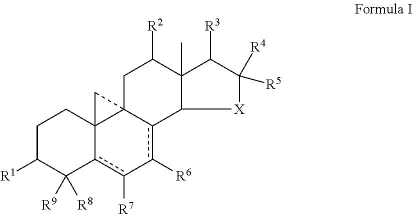

wherein
R¹ is —OH or —O—R¹⁰;
R² and R⁶ are independently —H or —OH;
   R³ is substituted or unsubstituted C₁-C₁₂ alkyl, substituted or unsubstituted C₂-C₁₂ alkenyl, or substituted or unsubstituted heterocycloalkyl groups,
   R⁴ is —H or —OH, or
   R³ and R⁴ taken together form a substituted or unsubstituted 5- or 6-membered cycloalkyl, heterocycloalkyl ring or spiro-heterocycloalkyl ring;
R⁵ is —H, —CH₂OH or —COOH;
R⁷ is —H, —OH or —O—R¹⁰;
R⁸ and R⁹ are independently —H or —CH₃;
R¹⁰ is a —C(═O)CH₃, or substituted or unsubstituted cycloalkyl or heterocycloalkyl groups; and
dashed bonds are independently present or absent.

In this formula, as in all structural formulas used hereinafter, it is understood that all carbon valences not shown here are satisfied by the groups illustrated and by hydrogen atoms.

An “alkyl” group refers to a saturated aliphatic hydrocarbon group. The alkyl group may be branched, straight chain, or cyclic (in which case, it would also be known as a “cycloalkyl” group) and may be substituted or unsubstituted. Depending on the structure, an alkyl group can be a monoradical or a diradical (i.e., an alkylene group). The alkyl group may have 1 to 12 carbon atoms, i.e., C₁-C₁₂, wherein the numerical range “1 to 12” refers to each integer in the given range; e.g., “1 to 12 carbon atoms” means that the alkyl group may have 1 carbon atom, 2 carbon atoms, 3 carbon atoms, etc., up to and including 12 carbon atoms. By way of example, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus, C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. The alkyl group could also be a “lower alkyl” having 1 to 6 carbon atoms.

The term “alkenyl” refers to a type of alkyl group in which at least two atoms of the alkyl group form a double bond. The alkenyl moiety may be branched or straight chain and optionally substituted. Non-limiting examples of an alkenyl group include —CH═CH₂, —C(CH₃)═CH₂, —CH═CHCH₃, —C(CH₃)═CHCH₃, and —CH(CH₃) CH═CHCH(CH₂CH₃) CH(CH₃) CH₃. Alkenyl groups could have 2 to 12 carbons. The alkenyl group could also be a “lower alkenyl” having 2 to 6 carbon atoms.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “cycloalkyl” refers to a monocyclic or polycyclic radical that contains only carbon and hydrogen, and may be saturated or partially unsaturated. Cycloalkyls of use in this invention have from 4 to 6 ring atoms and include, e.g., substituted and unsubstituted cyclobutyl, cyclopentyl and cyclohexyl.

As used herein, the term “non-aromatic heterocycle” or “heterocycloalkyl” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. Ideally heterocycloalkyl rings are composed of four, five, or six ring atoms. Heterocycloalkyl rings can be optionally substituted. Examples of heterocycloalkyls include, but are not limited to, lactams, lactones, cyclic imides, cyclic thioamides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidone, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. In certain embodiments, the heterocycloalkyl group is a substituted or unsubstituted tetrahydropyran or tetrahydrofuran.

The term “spiro-heterocycloalkyl” refers to saturated bicyclic ring system containing at least one heteroatom selected from oxygen, sulfur and nitrogen, in which the two rings are linked by a common atom. An exemplary spiro-heterocycloalkyl group is 5-azaspiro[2.3]hexanyl.

The term “optionally substituted” or “substituted” means that the referenced group may be substituted with one or more additional group(s) individually and independently selected from alkyl, cycloalkyl, aryl, heteroaryl, heteroalicyclic, alkylamine, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups. By way of example an optional substituent may be independently selected from —H, hydroxyl, oxo, C₁-C₄ alkyl (e.g., methyl or ethyl), C₃-C₆ cycloalkyl, heteroaryl, or heteroalkyl. Additional substituents are provided in the exemplary sterols disclosed herein.

Sitosterol has the following structure:

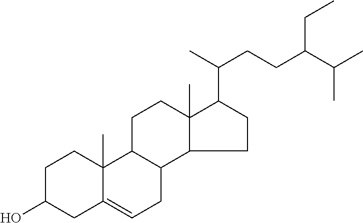 [SMILES: CCC(CCC(C)C1CCC2C3CC=C4CC(O)CCC4(C)C3CCC12C)C(C)C]

### (17-(4-ethyl-1,5-dimethyl-hexyl)-10,13-dimethyl-2,3,4,7,8,9,10,11,12,13,14,15,16,17-tetradecahydro-1H-cyclopenta[a]phenanthren-3-ol)

Isomers of sitosterol include α-sitosterol, β-sitosterol and γ-sitosterol, with β-sitosterol being the most common isomeric form of sitosterol.

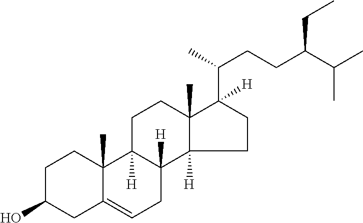

### β-sitosterol (3S,8S,9S,10R,13R,14S,17R)-17-((1R,4R)-4-ethyl-1,5-dimethyl-hexyl)-10,13-dimethyl-2,3,4,7,8,9,10,11,12,13,14,15,16,17-tetradecahydro-1H-cyclopenta[a]phenanthren-3-ol)

In certain embodiments, a structural analog of sitosterol refers to a compound falling within the scope of the structure of Formula II.

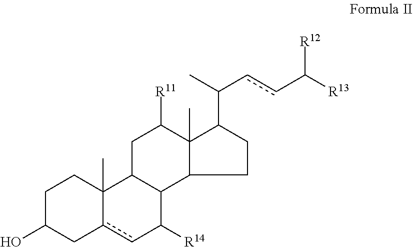

wherein
R¹¹ and R¹⁴ are independently —H or —OH;
R¹² is a lower alkyl or oxo group;
R¹³ is a lower alkyl, hydroxyl, or amine group; and
dashed bonds are independently present or absent.

For the purposes of this invention, the term “amine group” preferably refers to a group having the following structure:

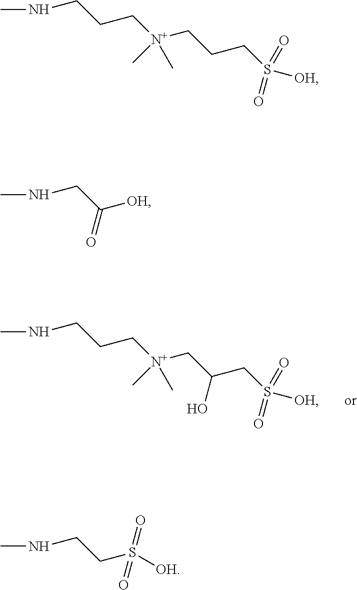

The sitosterol and structural analogs of the present invention can be obtained commercially, synthesized according to procedures known in the art, for example, as described by Hang (Hang, et al. (2010) Steroids, 75(12): 879-883) or obtained from a variety of natural sources such as nuts, seeds, fruits, vegetables, plant oils and dark chocolate.

To date, there is no disclosure in the art of the flavor use associated with individual sterols, let alone the specific flavor enhancement carbonation effect and alcohol sensation that a particular sterol provides.

It has now been shown that sitosterol and structural analogs are of use in enhancing carbonation effect in carbonated beverages and enhancing alcohol sensation in alcoholic consumables or alcohol-free beverages. In particular, β-sitosterol and structural analogs thereof are distinctly effective in enhancing carbonation effect in carbonated beverages. Further, β-sitosterol, is distinctly effective in enhancing alcohol sensation in alcoholic consumables or alcohol-free beverages. Thus, β-sitosterol and its structural analogs provide superior ingredient performance and possesses unexpected advantages in specific flavor enhancement.

Accordingly, one embodiment of the present invention relates to the finding of the unexpected effectiveness of sitosterol or an isomer thereof in enhancing carbonation effect in a carbonated beverage. Another embodiment of the present invention relates to the particular effectiveness of β-sitosterol and its structural analogs in enhancing carbonation effect in a carbonated beverage. Another embodiment of the present invention relates to the surprising finding of the unexpected effectiveness of sitosterol or an isomer or structural analog thereof in enhancing alcohol sensation in an alcoholic consumable or an alcohol-free beverage. Another embodiment of the present invention relates to the particular effectiveness of β-sitosterol in enhancing alcohol sensation in an alcoholic consumable or an alcohol-free beverage.

“Carbonation effect” refers to the pleasant, tingly sensation on the tongue created by the small bubbles of carbon dioxide released from a carbonated beverage. When pressure of a carbonated beverage is reduced, the dissolved carbon dioxide is expelled from the beverage as small bubbles. As a result, carbonic acid is formed in the oral mucosa and the small bubbles also cause tactile stimulation leading to carbonation effect including perceptions such as fizzy, tingling, burning and/or numbing.

A carbonated beverage is a beverage incorporated with carbon dioxide (CO₂). A carbonated beverage includes, for example, a carbonated water, a mineral water, a soda, a beer, a sparkling wine, a hard cider, a kombucha or a Champagne. Carbon dioxide is dissolved in the beverage under pressure. Alternatively, or additionally, a carbonate salt can be dissolved in the beverage, wherein the carbonate salt contributes carbon dioxide. In certain embodiments, a carbonated beverage contains carbon dioxide of a concentration greater than about 1 gram per liter (g/L), preferably from about 2 to about 10 g/L and more preferably from about 4 to about 8 g/L.

The term “carbonate salt” is understood to mean a salt comprising a cation and a bicarbonate anion (HCO₃⁻), a carbonate dianion (CO₃²⁻), or a combination thereof. A carbonate salt includes, for example, sodium bicarbonate (NaHCO₃), sodium carbonate (Na₂CO₃), potassium bicarbonate (KHCO₃), potassium carbonate (dipotassium carbonate, K₂CO₃), and a mixture thereof.

An alcoholic consumable is a food consumable that contains ethanol (CH₃CH₂OH), which includes, for example, an alcoholic beverage such as a distilled beverage, a wine, a beer or an alcohol-based fruit juice such as a cider, an alcoholic chocolate or an alcoholic candy. In certain embodiments, an alcoholic consumable contains ethanol in an amount greater than about 0.1 percent by volume, preferably from about 1 to about 50 percent by volume and more preferably from about 2 to about 40 percent by volume.

The terms “alcohol-free beverage” and “non-alcoholic beverage” are understood to mean the same, which is a non-alcoholic version of the alcoholic beverage defined in the above such as an alcohol-free distilled beverage or an alcohol-free beer. An alcohol-free beverage or a non-alcoholic beverage provides alcohol impression as an alcoholic beverage does. In certain embodiment, an alcohol-free beverage contains ethanol in an amount of 0.0 percent by volume.

“Alcohol sensation” or “alcohol impression” refers to alcoholic mouthfeel that includes perceptions such as astringency, dryness, heating, hot, tingling, irritating, numbing, burning and/or cooling.

The term “olfactory effective amount” is understood to mean the amount of sitosterol or an isomer or analog thereof used in an alcoholic consumable, wherein sitosterol or an isomer or analog thereof complements the alcohol sensation produced by ethanol, intensifies alcohol impression and therefore provides enhancement of alcohol sensation. In certain embodiments, the term “olfactory effective amount” is understood to mean the amount of β-sitosterol used in an alcoholic consumable.

The term “olfactory effective amount” is understood to mean the amount of sitosterol or an isomer or analog thereof used in a carbonated beverage, wherein sitosterol or an isomer or analog thereof intensifies the carbonation sensation produced by the carbonated beverage as compared to the same carbonated beverage, which lacks the sitosterol, isomer or analog. In certain embodiments, the term “olfactory effective amount” is understood to mean the amount of β-sitosterol or analog thereof used in a carbonated beverage. The term “olfactory effective amount” is also understood to mean the amount of sitosterol or an isomer or analog thereof used in an alcohol or alcohol-free beverage, wherein sitosterol or an isomer or analog thereof increases the alcohol impression and therefore provides enhancement of alcohol sensation. In other embodiments, the term “olfactory effective amount” is understood to mean the amount of β-sitosterol used in an alcohol-free beverage.

The olfactory effective amount may vary depending on many factors including other ingredients, their relative amounts and the olfactory effect that is desired. Any amount of sitosterol or an isomer or analog thereof that provides the desired degree of enhancement of alcohol sensation or enhancement of carbonation effect without exhibiting off-taste can be used.

In certain embodiments, the olfactory effective amount of sitosterol or an isomer or analog thereof employed in a carbonated beverage is about 1 part per trillion or greater by weight, preferably ranges from about 1 part per trillion to about 100 parts per million by weight, more preferably from about 1 part per billion to about 50 parts per million by weight and even more preferably from about 100 parts per billion to 10 parts per million by weight. The term “ppm” is understood to mean part per million by weight. The term “ppb” is understood to mean part per billion by weight. The term “ppt” is understood to mean part per trillion by weight.

In other embodiments, the olfactory effective amount of sitosterol or an isomer or analog thereof employed in an alcoholic consumable or an alcohol-free beverage is about 0.1 parts per billion or greater by weight, preferably ranges from about 0.1 parts per billion to about 5 parts per million by weight, more preferably from about 1 part per billion to about 1 part per million by weight and even more preferably from about 10 to about 250 parts per billion by weight.

Additional materials can also be used in conjunction with the compound of the present invention to encapsulate and/or deliver the flavor enhancement effect. Some well-known materials are, for example, but not limited to, polymers, oligomers, other non-polymers such as surfactants, emulsifiers, lipids including fats, waxes and phospholipids, organic oils, mineral oils, petrolatum, natural oils, perfume fixatives, fibers, starches, sugars and solid surface materials such as zeolite and silica.

The invention is described in greater detail by the following non-limiting examples.

### Example 1: Preparation of Test Samples

Materials were purchased from Aldrich Chemical Company unless noted otherwise.

A series of β-sitosterol solutions with concentrations ranging from 2.5 to 600 ppb were prepared in water. β-sitosterol exhibited no noticeable flavor itself.

Alcohol solutions containing varying amounts of ethanol (Alcohol by Volume, “ABV”) were prepared as follows:

Alcohol solutions at 1% ABV, 2% ABV, 2.5% ABV, 3% ABV and 5% ABV were prepared by adding varying amounts of ethanol in Brahma 0.0% alcohol-free beer (Anheuser-Busch InBev);

Alcohol solution at 6% ABV was prepared by adding ethanol in water; and

Alcohol solutions with ABV ranging from 8.75% to 50% were prepared by diluting CAPTAIN MORGAN® Original Spiced Rum (35% ABV) (Diageo North America Inc.) in varying amounts of water.

Carbonated water containing carbon dioxide at 6.0, 7.2 and 8.0 g/L, respectively, were also prepared.

### Example 2: Enhancement of Alcohol Sensation in Alcohol-Free Beverages

A β-sitosterol solution (prepared in Example 1) was added in Brahma 0.0% alcohol-free beer to yield a final concentration of 25 ppb. The alcohol impression of the obtained sample was evaluated and compared by a panel with alcohol solutions at 1% ABV, 2% ABV, 2.5% ABV, 3% ABV and 5% ABV (prepared in Example 1). β-Sitosterol (25 ppb) provided an equal level of alcohol impression as the alcohol solution at 2.5% ABV. Accordingly, β-Sitosterol provided alcohol sensation in alcohol-free beverages.

### Example 3: Enhancement of Alcohol Sensation in Alcoholic Beverages

Enhanced Alcohol Sensation by β-Sitosterol.

A β-sitosterol solution (prepared in Example 1) was added to the alcohol solution at 6% ABV (prepared in Example 1) to yield a final concentration of 100 ppb. The alcohol sensation of the obtained sample was evaluated and compared by a panel with the alcohol solution at 6% ABV using an intensity scale of 0 to 10, where 0=none, 4=medium, 7=high and 10=extremely high. The intensity of alcohol sensation was rated. Mean (“Alcohol Intensity”) and standard error of the mean (“SE”, ±) were obtained (Table 1).

TABLE 1

| Sample | Mean Alcohol Intensity ± SE |
|---|---|
| Solution of 6% ABV | 4.38 ± 0.32 |
| Solution of 6% ABV + 100 ppb | 7.21 ± 0.27 |
| β-sitosterol | |

The difference of alcohol intensity between the two groups was statistically significant (p<0.01). Thus, β-sitosterol was effective in enhancing alcohol sensation in alcoholic beverages.

Enhanced Alcohol Sensation with Varying Amounts of β-Sitosterol.

A series of β-sitosterol solutions (prepared in Example 1) were added to the alcohol solution at 17.5% ABV (prepared in Example 1) to yield final concentrations of β-sitosterol ranging from 3 to 500 ppb. The alcohol sensation of the obtained samples containing different amounts of β-sitosterol was evaluated. This analysis indicated that at 3 ppb, β-sitosterol provided a slight but noticeable increase in alcohol sensation. At higher concentrations, β-sitosterol produced a clear increase in alcohol intensity. However, at 200 ppb, the enhancement of alcohol sensation of β-sitosterol reached a stable plateau.

Enhanced Alcohol Sensation with Varying Amounts Alcohol.

A β-sitosterol solution (prepared in Example 1) was added to alcohol solutions at 8.75% ABV, 17.5% ABV and 21.75% ABV (prepared in Example 1), respectively, to yield a final concentration of 25 ppb β-sitosterol. The alcohol sensation of the obtained samples was evaluated and compared by a panel with alcohol solutions at higher levels of ABV. This analysis indicated that an alcohol solution at 8.75% ABV with added β-sitosterol exhibited an alcohol sensation at the level of an alcohol solution at 10% ABV; an alcohol solution at 17.5% ABV with added β-sitosterol exhibited an alcohol sensation at the level of an alcohol solution at 22.5% ABV; and an alcohol solution at 21.75% ABV with added β-sitosterol exhibited an alcohol sensation at the level of an alcohol solution at 35% ABV.

### Example 4: Enhancement of Carbonation Effect in Carbonated Beverages

Enhanced Carbonation Effect by β-Sitosterol.

A β-sitosterol solution (prepared in Example 1) was added to the carbonated water (7.2 g/L CO₂) (prepared in Example 1) to yield a final concentration of 2.5 ppt. The carbonation effect of the obtained sample was evaluated and compared by a panel with the carbonated water using an intensity scale of 0 to 10, where 0=none, 4=medium, 7=high and 10=extremely high. The intensity of carbonation effect was rated. Mean (“Carbonation Intensity”) and standard error of the mean (“SE”, ±) were obtained (Table 2).

TABLE 2

| | Mean Carbonation |
|---|---|
| Sample | Intensity ± SE |
| Solution of 7.2 g/L CO₂ | 4.88 ± 2.53 |
| Solution of 7.2 g/L CO₂ + 2.5 ppt | 7.19 ± 1.73 |
| β-sitosterol | |

The difference of carbonation intensity between the two groups was statistically significant (p<0.01). Thus, β-sitosterol was effective in enhancing carbonation effect in carbonated beverages.

Enhanced Carbonation Effect with Varying Amounts of β-Sitosterol.

A series of β-sitosterol solutions (prepared in Example 1) were added to carbonated water (7.2 g/L CO₂) (prepared in Example 1) to yield final concentrations ranging from 0.1 ppt to 50 ppt. The carbonation sensation of the obtained samples containing different amounts of β-sitosterol was evaluated. This analysis indicated that at 0.1 ppt, β-sitosterol provided a slight increase in carbonation perception. At 2.5 ppt, β-sitosterol produced a clear increase in carbonation perception. At a level of 5 ppt or higher, the enhancement of carbonation effect by β-sitosterol reached a stable plateau.

Enhanced Carbonation Effect with Varying Amounts of Carbonation.

A β-sitosterol solution (prepared in Example 1) was added to carbonated water containing 6.0, 7.2 or 8.0 g/L CO₂ (prepared in Example 1), respectively, to yield a final concentration of 2.5 ppt β-sitosterol. The carbonation sensation of the obtained samples was evaluated. In all samples containing different amounts of CO₂, β-sitosterol provided enhancement of carbonation sensation.

### Example 5: Structural Analogs of β-Sitosterol

Among the numerous sterols, some of the most commonly known ones include, for example, sitosterol, campesterol, stigmasterol, brassicasterol and ergosterol. Sitosterol and its structural analogs are set forth below. Those with skill in the art many of these structural analogs are readily available and can exhibit unexpected and significant differences in properties and functions.

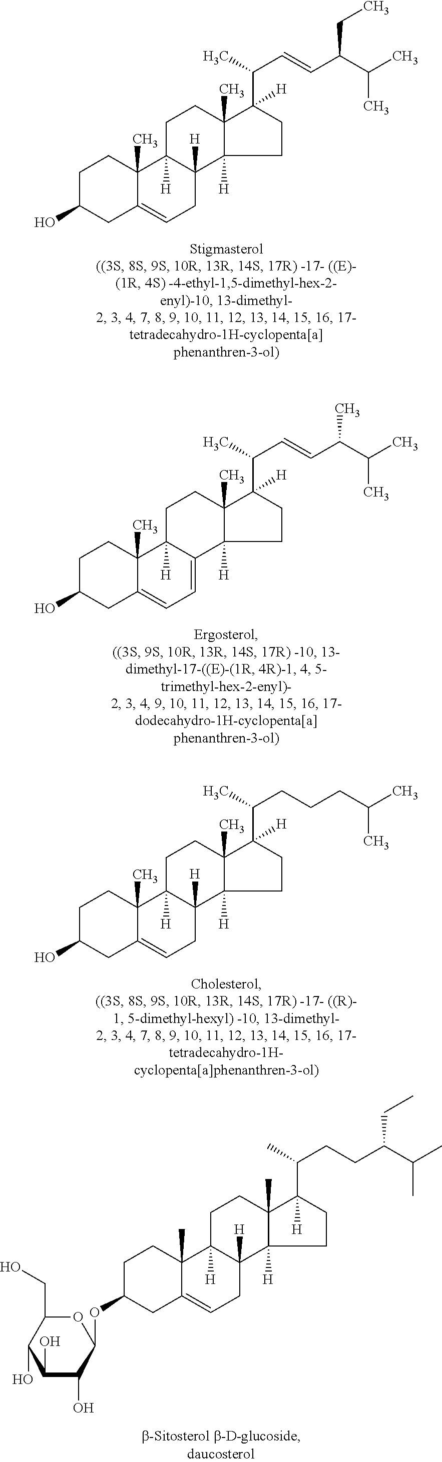

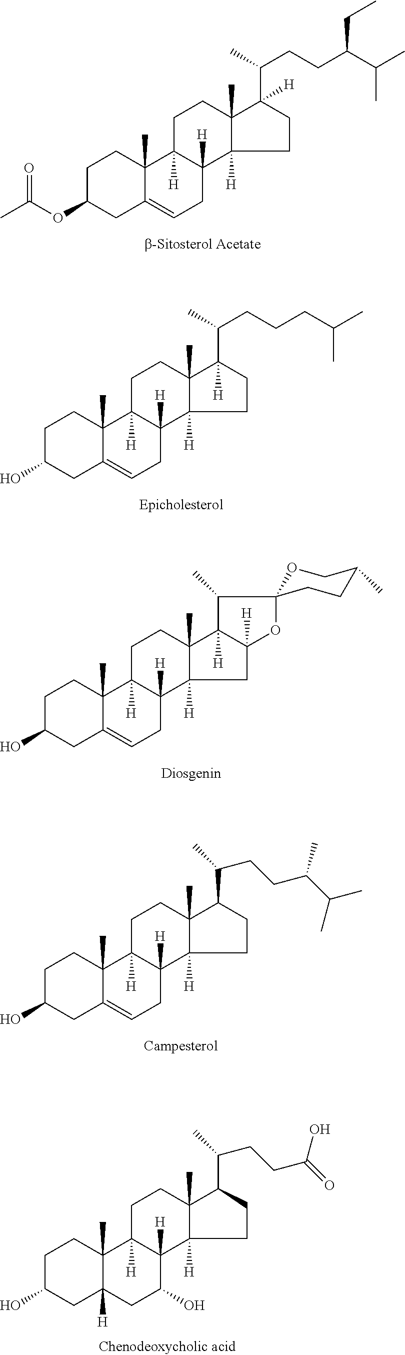

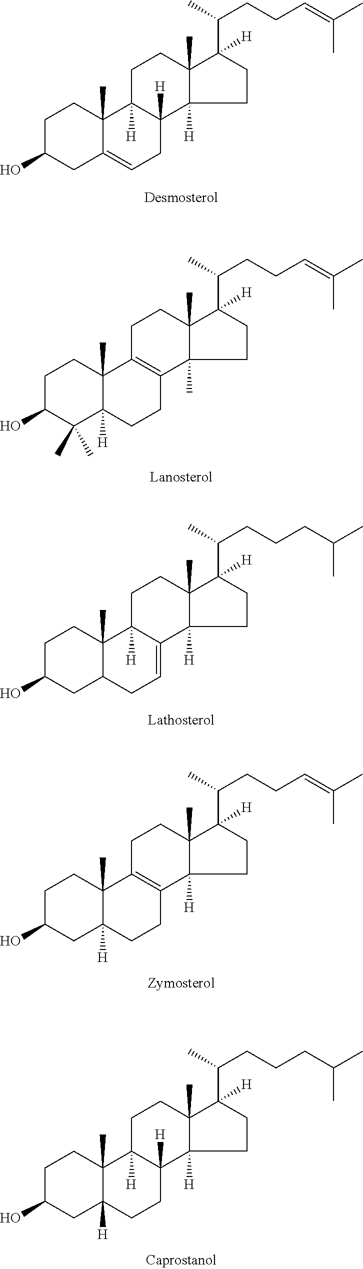

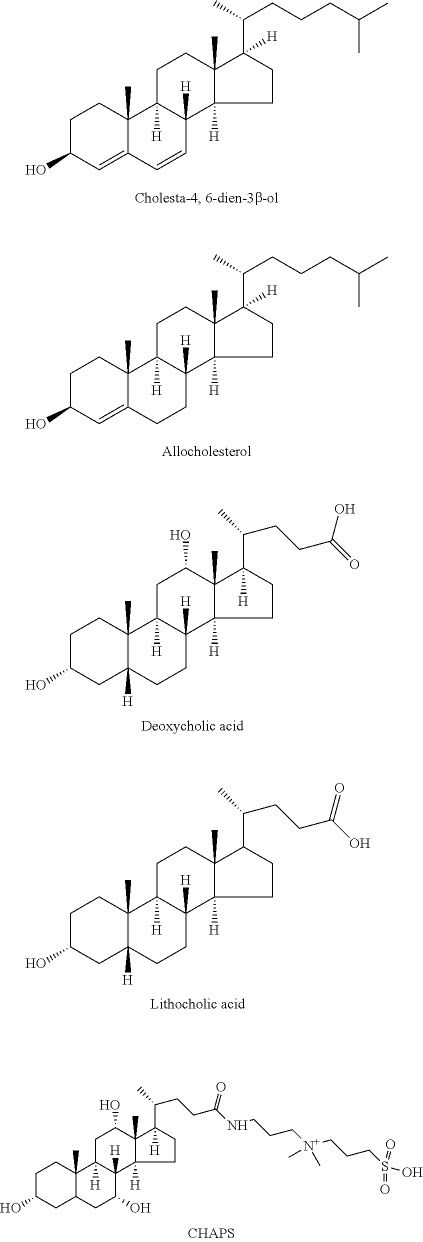

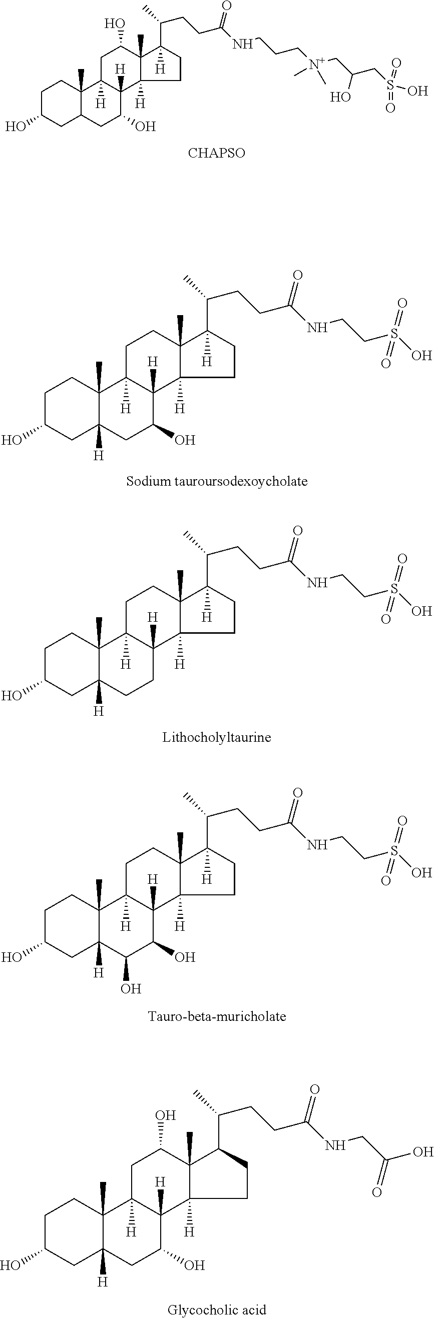

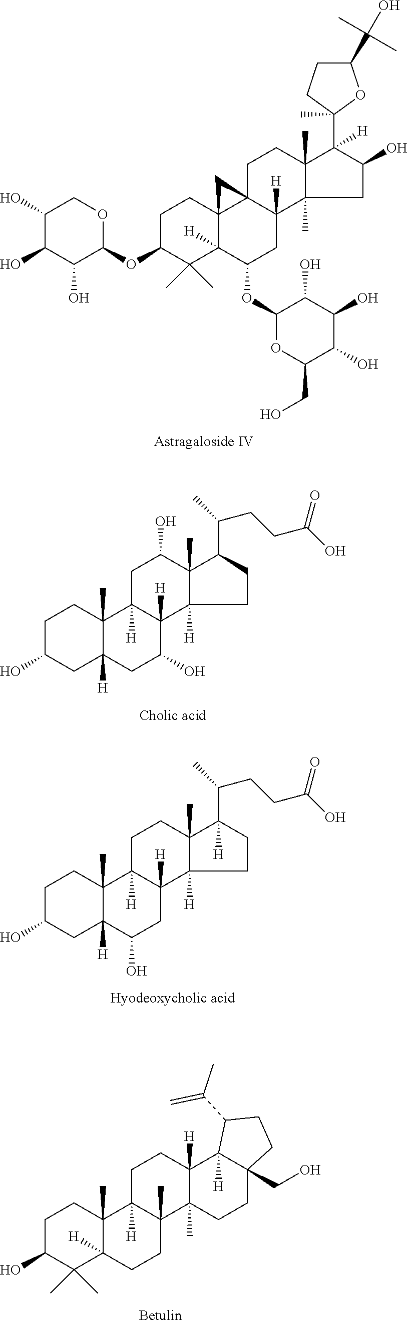

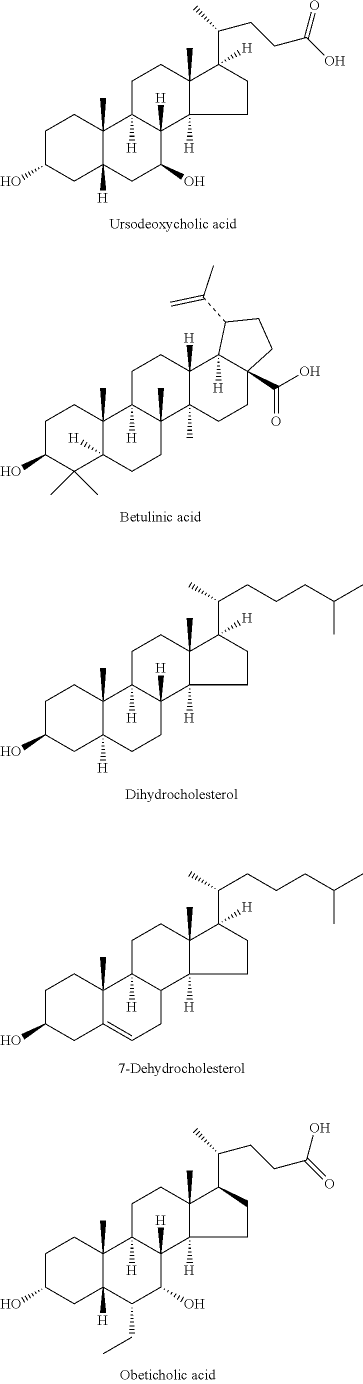

### Example 6: Enhancement of Carbonation Effect in Carbonated Beverages Using β-Sitosterol Structural Analogs

Selected structural analogs of sitosterol were tested for their ability to enhance the carbonation effect of a carbonated beverage. The analogs were used in amounts of 400 ppb and 200 ppb to 5 ppm. The sensory evaluation was carried by a trained panel that tasted each of the compounds in a lime-flavored carbonated water (Vintage seltzer water). The intensity of carbonation effect was rated and compared to other samples (Table 3)

TABLE 3

| Sample | Carbonation Effect |
|---|---|
| β-Sitosterol β- | 200 ppb-5 ppm: Slight tingly tip of tongue, |
| D-glucoside, | numbing, very slight foamy, feel bubbles only |
| Daucosterol | on tip of tongue. Like a weaker version of β- |
| | sitosterol. |
| Cholesterol | 200 ppb-5 ppm: Very similar to Deoxycholic |
| | acid. Still works but is foamy, does not add a |
| | tingly and more bubbly characteristic. |
| Diosgenin | 200 ppb-5 ppm: Not much effect on carbonation, |
| | this analog found to have the least effect. |
| Stigmasterol | 200 pb-5 ppm: 5 ppm is a good level, adds more |
| | aroma, adds more upfront bubbly (not as violent |
| | as Chenodeoxycholic acid) but then it changes |
| | into like a β-Sitosterol foamy tingly, this |
| | molecule is closest to β-Sitosterol, tingly. |
| Chenodeoxycholic | 200 ppb-5 ppm: Adds more violent upfront |
| acid | bubbling, works, very good, spotty, 200 ppb |
| | preferred by panel, clean, maintains profile, |
| | upfront is obvious, has mouthfeel, not that |
| | penetrating, not pungent, cleaner than β- |
| | Sitosterol, β-Sitosterol is more tingly than |
| | this molecule, sharp. |
| Deoxycholic acid | 200 ppb-5 ppm: Works, makes bubbles more |
| | foamy, no increase in tingly, higher dosage |
| | levels are more foamy. |
| CHAPS | 200 ppb-5 ppm: Foamy but better than |
| | Deoxycholic acid and Cholesterol, has |
| | mouthcoating characteristic that makes bubbles |
| | lie and pop on your tongue but also the bubbles |
| | fill the whole mouth. Is more dispersed |
| | compared to other analogs, adds slightly more |
| | bubbly, full mouthfeel characteristic, much |
| | different than Chenodeoxycholic acid, close to |
| | β-Sitosterol but not as much as Stigmasterol. |
| Sodium | 200 ppb: Very impactful, numbing, slightly more |
| taurourso- | foamy than control, more flavor impact, slight |
| deoxycholate | sour and has like a shock effect on tongue, |
| | makes bubbles feel larger than control, |
| | carbonation everywhere in mouth, has |
| | astringency may be due to sourness, |
| | Chenodeoxycholic acid may be slightly stronger |
| | upfront in carbonation. In the bottle, bubbles |
| | very apparent and did not go away. |
| Glycocholic acid | 200 ppb: Longer lasting bubbles than control, |
| hydrate | not huge effect, more tiny bubbles, somewhat |
| | similar to β-Sitosterol, more foam than |
| | control, more flavor impact, carbonation |
| | builds, delayed carbonation effect, mild, would |
| | be good for champagne, overall makes |
| | carbonation last longer with smaller bubbles. |
| Lithocholic acid | 200 ppb: More upfront carbonation, slightly |
| | working not huge impact, weaker than |
| | Glycocholic acid and Sodium |
| | tauroursodeoxycholate. |
| | 400 ppb: Works, foamy at the end, sharpness, |
| | sour, different than Chenodeoxycholic acid, |
| | more closer to β-Sitosterol, works with lime |
| | flavor, longer tingle, stronger bubbles. |
| Cholic acid | 200 ppb: More tingly bubbles on tongue, linger, |
| | slight lingering burn, provides more body, |
| | somewhat weak, sharper burn. |
| | 400 ppb: Very foamy, foam builds, lingering |
| | numbing, long lasting, almost bitter lingering, |
| | long lasting, needle like foams. |
| Astragaloside IV | 200 ppb: More impactful, long lasting, very |
| | sharp, burn upfront, burn tip of tongue, strong |
| | tongue penetration compared to Cholic acid and |
| | Hyodeoxycholic acid, foamy. |
| Hyodeoxycholic | 200 ppb: Very foamy, fast bubbles upfront, |
| acid | increasing number of bubbles, foamy finish, |
| | slightly weak but has increase in number of |
| | bubbles. |
| Betulin | 200 ppb: Long lasting popping, sour, works with |
| | lime, strong burn, penetrating feeling. Sharp |
| | up front, pungent, all over the mouth |
| Ursodeoxycholic | 200 ppb: Foamy, fills up whole mouth, some |
| acid | detergent like taste, bubbles spread, smaller |
| | bubbles, most numbing compared to Betulin and |
| | Betulinic acid, slightly bitter. |
| Betulinic acid | 200 ppb: Cooling, almost like menthol, |
| | bubbling, numbing, short time of bubble |
| | sensation, small bubbles, slightly weaker than |
| | Betulin and Ursodeoxycholic acid. |

What is claimed is:

1. A method of enhancing carbonation effect in a carbonated beverage comprising the step of adding an olfactory effective amount of a compound of Formula I: 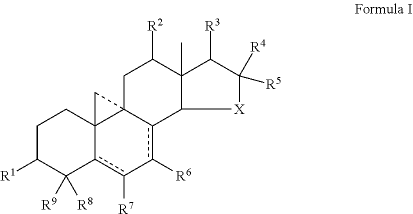
wherein
X is —C— or —C—C—;
R¹ is —OH or —O—R¹⁰;
R² and R⁶ are independently —H or —OH;
R³ is a substituted or unsubstituted C₁-C₁₂ alkyl, substituted or unsubstituted C₂-C₁₂ alkenyl, or substituted or unsubstituted heterocycloalkyl group,
R⁴ is —H or —OH, or
R³ and R⁴ taken together form a substituted or unsubstituted 5- or 6-membered cycloalkyl, heterocycloalkyl or spiro-heterocycloalkyl ring;
R⁵ is —H, —CH₂OH or —COOH;
R⁷ is —H, —OH or —O—R¹⁰;
R⁸ and R⁹ are independently —H or —CH₃;
R¹⁰ is a —C(═O)CH₃, or substituted or unsubstituted cycloalkyl or heterocycloalkyl group; and
dashed bonds are independently present or absent.

2. The method of claim 1, wherein the compound has the structure of Formula II: 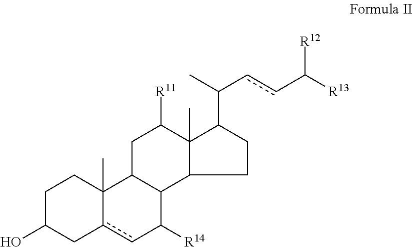
wherein
R¹¹ and R¹⁴ are independently —H or —OH;
R¹² is a lower alkyl or oxo group;
R¹³ is a lower alkyl, hydroxyl, or amine group; and
dashed bonds are independently present or absent.

3. The method of claim 1, wherein the compound is β-sitosterol, stigmasterol, chenodeoxycholic acid or CHAPS.

4. The method of claim 1, wherein the olfactory effective amount is about 1 part per trillion or greater by weight.

5. The method of claim 1, wherein the olfactory effective amount is from about 1 part per billion to about 100 parts per million by weight.

6. The method of claim 1, wherein the carbonated beverage is selected from the group consisting of a carbonated water, a mineral water, a soda, a beer, a sparkling wine and a Champagne.

7. A carbonated beverage comprising an olfactory effective amount of a compound of Formula I: 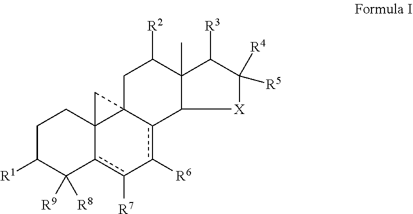
wherein
X is —C— or —C—C—;
R¹ is —OH or —O—R¹⁰;
R² and R⁶ are independently —H or —OH;
R³ is a substituted or unsubstituted C₁-C₁₂ alkyl, substituted or unsubstituted C₂-C₁₂ alkenyl, or substituted or unsubstituted heterocycloalkyl group,
R⁴ is —H or —OH, or
R³ and R⁴ taken together form a substituted or unsubstituted 5- or 6-membered cycloalkyl, heterocycloalkyl or spiro-heterocycloalkyl ring;
R⁵ is —H, —CH₂OH or —COOH;
R⁷ is —H, —OH or —O—R¹⁰;
R⁸ and R⁹ are independently —H or —CH₃;
R¹⁰ is a —C(═O)CH₃, or substituted or unsubstituted cycloalkyl or heterocycloalkyl groups; and
dashed bonds are independently present or absent.

8. The carbonated beverage of claim 7, wherein the compound has the structure of Formula II: 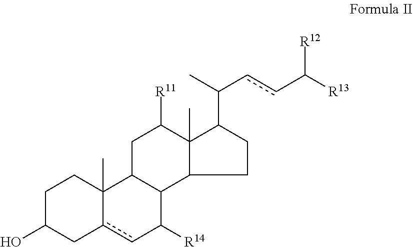
wherein
R¹¹ and R¹⁴ are independently —H or —OH;
R¹² is a lower alkyl or oxo group;
R¹³ is a lower alkyl, hydroxyl, or amine group; and
dashed bonds are independently present or absent.

9. The carbonated beverage of claim 7, wherein the compound is R-sitosterol, stigmasterol, chenodeoxycholic acid or CHAPS.
